(19) United States
(12) Patent Application Publication
(10) Pub. No.: US 2018/0252665 A1
(43) Pub. Date: Sep. 6, 2018

(54) SYSTEM AND METHODS FOR THE DETECTION OF BIOMARKERS OF NEURODEGENERATIVE DISORDERS

(71) Applicant: CASE WESTERN RESERVE UNIVERSITY, Cleveland, OH (US)

(72) Inventors: Chung Chiun Liu, Cleveland Heights, OH (US); Yifan Dai, Cleveland Heights, OH (US)

(21) Appl. No.: 15/970,738

(22) Filed: May 3, 2018

Related U.S. Application Data
(63) Continuation-in-part of application No. PCT/US2018/025275, filed on Mar. 29, 2018.
Application No. PCT/US2018/025275 is a continuation-in-part of application No. 15/314,393, filed on Nov. 28, 2016.
(60) Provisional application No. 62/500,875, filed on May 3, 2017.
(60) Provisional application No. 62/478,138, filed on Mar. 29, 2017.
(60) Provisional application No. 62/003,221, filed on May 27, 2014.

(51) Int. Cl. G01N 27/327; G01N 33/543; G01N 33/68; C07K 16/18

(52) U.S. Cl. CPC G01N 27/3277; G01N 33/5438; G01N 2333/47; C07K 16/18; G01N 2333/4709; G01N 33/6896

(57) ABSTRACT

A sensor for the detection of β-amyloid 42, T-Tau, and/or P-Tau in a sample includes a substrate, a working electrode and counter electrode formed on a surface of the substrate, and an anti-β-amyloid 42 antibody, anti-T-Tau antibody, and/or anti-P-Tau antibody functionalized or chemically functionalized to a surface of an exposed portion of the working electrode.

### RELATED APPLICATION

This application claims priority from U.S. Provisional Application No. 62/500,875, filed May 3, 2017, and is a Continuation-in-Part of PCT Application No. PCT/US2018/025275, filed Mar. 29, 2018, which claims priority to U.S. Provisional Application Ser. No. 62/478,138, filed Mar. 27, 2017, this application is also Continuation-in-Part of U.S. application Ser. No. 15/314,393, filed Nov. 28, 2016, which is a 371 filing of PCT/US2015/032609, filed May 27, 2015, which claims priority to U.S. Provisional Application Ser. No. 62/003,221, filed May 27, 2014, the subject matter of which are incorporated herein by reference in their entirety.

### BACKGROUND

Neurological degenerative disorders are a frightening health burden. These disorders include Alzheimer's disease (AD), traumatic brain injury (TBI), different forms of dementia including Parkinson's disease, and Creutzfeldt-Jacob disease, among others. Both the physical and socio-economic burden of neuro-degenerative disorders are immense. For example, there were currently 5.2 million Americans living with Alzheimer's disease in 2016, and this number will increase to 23.8 million in 2050. The cost of caring for Alzheimer's patients in the U.S. is estimated to be $236 billion in 2016. Furthermore, over 40% of family caretakers report that the emotional stress of their role is high to very high. TBI is a major cause of long-term disability, affecting 100,000 individuals in the U.S. annually. There are no officially established diagnostic methods for TBI. The annual cost of TBI in U.S. is estimated to be $48.3 billion, including $31.7 billion spent on hospital costs, while $16.6 billion goes toward costs associated with fatalities.

Researchers have scientifically assessed amyloidopathy and tauopathy and their effects on neurodegenerative disorders. In amyloidopathy, the formation and characterization of β-amyloid 42 biomolecules are of significance. In tauopathy, the microtubule associate protein, Tau, which binds to and stabilizes microtubules axons, is the protein of focus. The levels of total Tau (T-Tau) and the phosphorylated Tau (P-Tau) are considered useful biomarkers for assessing neurodegenerative disorders. It is generally agreed that any additional tools for the detection of these biomolecules, β-amyloid 42, T-Tau, and P-Tau, will be very useful in evaluating neurodegenerative disorders. Tau protein is expressed in the neurons of central nervous system and it is critically important in axonal maintenance and axonal transport. T-Tau and P-Tau levels in brain, blood, and cerebrospinal fluid related to neuro-degenerative disorders are well recognized.

Cerebrospinal fluid (CSF) is commonly chosen as the physiological fluid test medium for the assessment of β-amyloid 42, T-Tau, P-Tau, and others. CSF provides good information for neurological related phenomena, but the collection of CSF is an elaborate and complicated process. On the other hand, the collection of a small blood sample (20-30 L) is a minimally invasive procedure and can be administered relatively simply. Measurements of Tau level in blood as a reliable biomarker for Alzheimer's disease have been postulated and reported. The technical challenge will be providing additional tools for the detection of these biomarkers in a simple and accurate manner.

### SUMMARY

Embodiments described herein relate to a detection system, method, and in vitro assay for detecting, identifying, quantifying, and/or determining biomarkers of neurodegenerative disorders, such as Alzheimer's disease, traumatic brain injury, and Creutzfeldt-Jacob disease, in a bodily sample of a subject as well as to a detection system, method, and in vitro assay for diagnosing, identifying, staging, and/or monitoring neurodegenerative disorders, such as Alzheimer's disease, traumatic brain injury, and Creutzfeldt-Jacob disease, in a subject having, suspected of having a neurodegenerative disorders, such as Alzheimer's disease, traumatic brain injury, and Creutzfeldt-Jacob disease.

In some embodiments, the system and/or method for detecting, indentifying, quantifying, and/or determining neurodegenerative disorders can detect, indentify, quantify, and/or determine the amount or level of β-amyloid 42, T-Tau, and/or P-Tau in a bodily sample. The system can include an electrochemical biosensor, for detecting, identifying, quantifying, and/or determining the amount or level of β-amyloid 42, T-Tau, P-Tau in a sample, such as blood or other fluids (e.g., cerebrospinal fluid). The system and method described herein can provide a single use, disposable, and cost-effective means for simple assessment of β-amyloid 42, T-Tau, and/or P-Tau in bodily samples obtained by non-invasive or minimally invasive means.

In some embodiments, the system and methods described herein includes an electrochemical biosensor, a redox solution, and a measuring device. The electrochemical biosensor can produce a signal that is related to the presence or quantity of the β-amyloid 42, T-Tau, and/or P-Tau being detected in a sample. In some embodiments, the system can be used to detect and/or quantify β-amyloid 42, T-Tau, and/or P-Tau that is present in blood or a biological fluid, such as cerebrospinal fluid.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. An anti-β-amyloid 42 antibody, anti-T-Tau antibody, and/or anti-P-Tau antibody can be functionalized or chemically functionalized to a surface of the exposed portion of the working electrode. The anti-β-amyloid 42 antibody, anti-T-Tau antibody, and/or anti-P-Tau antibody selectively binds to, respectively, β-amyloid 42, T-Tau, and/or P-Tau in a sample, and the β-amyloid 42, T-Tau, and/or P-Tau once bound is detectable by measuring the current flow between the working electrode and counter electrode.

The redox solution is applied to the working electrode for determining the quantity of β-amyloid 42, T-Tau, and/or P-Tau in the sample bound to the anti-β-amyloid 42, anti-T-Tau, and/or anti-P-Tau antibody. The measuring device applies voltage potentials to the working electrode and counter electrode and measures the current flow between the working electrode and counter electrode to determine the level of the β-amyloid 42, T-Tau, and/or P-Tau in a bodily sample, such as blood or cerebrospinal fluid.

In some embodiments, the working electrode and the counter electrode include metalized films. The metalized films used to form the working electrode and the counter electrode can independently comprise gold, platinum, palladium, silver, carbon, alloys thereof, and composites thereof. The metalized films can be provided on the surface of the substrate by sputtering or coating the films on the surface and then laser ablating the films to form the working electrode and counter electrode.

In other embodiments, the sensor can include a reference electrode on the surface of the substrate. The dielectric can cover a portion of the reference electrode.

In other embodiments, the anti-β-amyloid 42 antibody, anti-T-Tau antibody, and/or anti-P-Tau antibody can be chemically functionalized to the surface of the working electrode coated with a 3-mercaptopropionic acid (MPA) monolayer.

### BRIEF DESCRIPTION OF THE DRAWINGS

FIG. 1 is a schematic illustration of a biosensor in accordance with an aspect of the application.

FIG. 2 illustrates structures and dimensions of the thin film gold-based T-Tau biosensor in accordance with an antibody described herein.

FIGS. 3(A-B) illustrate (A) Cyclic voltammograms obtained in a solution of K₃Fe(CN)₆ and K₄Fe(CN)₆, 5 mM in each component, having 0.1 M KCl at scan rates ranging from 30 to 100 mV/s and (B) oxidation peak current plotted versus square root of scan rate.

FIG. 4 illustrates nyquist plots obtained from EIS in presence of K₃Fe(CN)₆/K₄Fe(CN)₆ redox couple for the two groups of sensors (group 1 subjected to cleaning procedure), and equivalent Randles circuit.

FIG. 5 illustrates electrochemical impedance spectroscopy (EIS) Nyquist plots obtained in presence of K₃Fe(CN)₆/K₄Fe(CN)₆ redox couple for antibody immobilized biosensors incubated in T-Tau solutions for 3 h at room temperature. Antigen solutions were prepared in 0.1 M PBS.

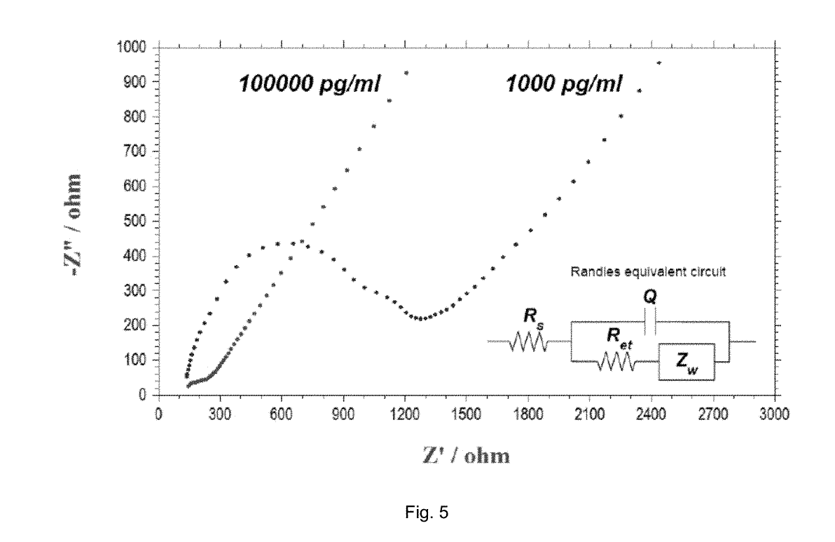

FIGS. 6(A-B) illustrate (A) Differential pulse voltammetry (DPV) measurement of T-Tau proteins over the concentration range of 1,000 pg/mL to 100,000 pg/mL in 0.1 M PBS solution. (B) Calibration curve of the DPV outputs and T-Tau protein concentration in 0.1 M PBS solution. Anti-T-Tau concentration is 500,000 pg/mL.

FIGS. 7(A-B) illustrate (A) DPV measurement of T-Tau proteins over the concentration range of 1,000 pg/mL to 100,000 pg/mL in undiluted human serum. (B) Calibration curve obtained from DPV measurements for T-Tau protein concentration range of 1000 to 1,000,000 pg/mL in undiluted human serum. Anti-TTau concentration is 500,000 pg/mL.

FIG. 8 illustrates interference study using β-amyloid 42 of 50,000 pg/mL as the biomarker.

### DETAILED DESCRIPTION

Unless specifically addressed herein, all terms used have the same meaning as would be understood by those of skilled in the art of the subject matter of the application. The following definitions will provide clarity with respect to the terms used in the specification and claims.

As used herein, the term “monitoring” refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication.

As used herein, the term “quantitative data” or “quantitative level” or “quantitative amount” refers to data, levels, or amounts associated with any dataset components (e.g., markers, clinical indicia,) that can be assigned a numerical value.

As used herein, the term “subject” refers to a human or another mammal. Typically, the terms “subject” and “patient” are used herein interchangeably in reference to a human individual.

As used herein, the term “bodily sample” refers to a sample that may be obtained from a subject (e.g., a human) or from components (e.g., tissues) of a subject. The sample may be of any biological tissue or fluid with, which analytes described herein may be assayed. Frequently, the sample will be a “clinical sample”, i.e., a sample derived from a patient. Such samples include, but are not limited to, bodily fluids, e.g., saliva, breath, urine, blood, cerebrospinal fluid, plasma, or sera; and archival samples with known diagnosis, treatment and/or outcome history. The term biological sample also encompasses any material derived by processing the bodily sample. Processing of the bodily sample may involve one or more of, filtration, distillation, extraction, concentration, inactivation of interfering components, addition of reagents, and the like.

As used herein, the terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal.

As used herein, the terms “normal” and “healthy” are used interchangeably. They refer to an individual or group of individuals who have not shown any symptoms of neurodegenerative disorder, and have not been diagnosed with neurodegenerative disorder. Preferably, the normal individual (or group of individuals) is not on medication affecting a neurodegenerative disorder. In certain embodiments, normal individuals have similar sex, age, body mass index as compared with the individual from which the sample to be tested was obtained. The term “normal” is also used herein to qualify a sample isolated from a healthy individual.

As used herein, the terms “control” or “control sample” refer to one or more biological samples isolated from an individual or group of individuals that are normal (i.e., healthy). The term “control”, “control value” or “control sample” can also refer to the compilation of data derived from samples of one or more individuals classified as normal, and/or one or more individuals diagnosed with a neurodegenerative disorder.

As used herein, the term “indicative of neurodegenerative disorder”, when applied to an amount of at least one of β-amyloid 42, T-Tau, and/or P-Tau in a bodily sample, refers to a level or an amount, which is diagnostic of neurodegenerative disorder such that the level or amount is found significantly more often in subjects with the disorder than in subjects without the neurodegenerative disorder (as determined using routine statistical methods setting confidence levels at a minimum of 95%). Preferably, a level or amount, which is indicative of a neurodegenerative disorder, is found in at least about 60% of subjects who have the neurodegenerative disorder and is found in less than about 10% of subjects who do not have the neurodegenerative disorder. More preferably, a level or amount, which is indicative of neurodegenerative disorder, is found in at least about 70%, at least about 75%, at least about 80%, at least about 85%, at least about 90%, at least about 95% or more in subjects who have the neurodegenerative disorder and is found in less than about 10%, less than about 8%, less than about 5%, less than about 2.5%, or less than about 1% of subjects who do not have the neurodegenerative disorder.

As used herein, the term “neurodegenerative disorder” is intended to include neurological disorders to the nervous system including Parkinson's disease, Dementia Pugilistica, Huntington's disease, Alzheimer's disease, Creutzfeldt-Jakob disease, brain injuries secondary to seizures which are induced by radiation, exposure to ionizing or iron plasma, nerve agents, cyanide, toxic concentrations of oxygen, neurotoxicity due to central nervous system (CNS) malaria or treatment with anti-malaria agents, trypanosomes, malarial pathogens, and other CNS traumas.

Embodiments described herein relate to a detection system, method, and in vitro assay for detecting, identifying, quantifying, and/or determining biomarkers of neurodegenerative disorders, such as Alzheimer's disease, traumatic brain injury, and Creutzfeldt-Jacob disease, in a bodily sample of a subject as well as to a detection system, method, and in vitro assay for diagnosing, identifying, staging, and/or monitoring neurodegenerative disorders, such as Alzheimer's disease, traumatic brain injury, and Creutzfeldt-Jacob disease, in a subject having, suspected of having a neurodegenerative disorders, such as Alzheimer's disease, traumatic brain injury, and Creutzfeldt-Jacob disease.

In some embodiments, the system and/or method for detecting, indentifying, quantifying, and/or determining neurodegenerative disorders can detect, indentify, quantify, and/or determine the amount or level of β-amyloid 42, T-Tau, and/or P-Tau in a bodily sample. The system can include an electrochemical biosensor, for detecting, identifying, quantifying, and/or determining the amount or level of β-amyloid 42, T-Tau, P-Tau in a bodily sample, such as blood or other fluids (e.g., cerebrospinal fluid). The system and method described herein can provide a single use, disposable, and cost-effective means for simple assessment of β-amyloid 42, T-Tau, and/or P-Tau in bodily samples obtained by non-invasive or minimally invasive means.

In some embodiments, the electrochemical biosensor includes a substrate, a working electrode formed on a surface of the substrate and a counter electrode formed on the surface of the substrate. A dielectric layer covers a portion of the working electrode and counter electrode and defines an aperture exposing other portions of the working electrode and counter electrode. An anti-β-amyloid 42 antibody, anti-T-Tau antibody, and/or anti-P-Tau antibody is functionalized or chemically functionalized to a surface of the exposed portion of the working electrode. The anti-β-amyloid 42 antibody, anti-T-Tau antibody, and/or anti-P-Tau antibody selectively binds to respectively β-amyloid 42, T-Tau, and/or P-Tau in a sample, and the β-amyloid 42, T-Tau, and/or P-Tau once bound is detectable by measuring the current flow between the working electrode and counter electrode.

The redox solution is applied to the working electrode for determining the quantity of β-amyloid 42, T-Tau, and/or P-Tau in the sample bound to the anti-β-amyloid 42, anti-T-Tau, and/or anti-P-Tau antibody. The measuring device applies voltage potentials to the working electrode and counter electrode and measures the current flow between the working electrode and counter electrode to determine the level of the β-amyloid 42, T-Tau, and/or P-Tau in a sample, such as a blood or cerebrospinal fluid.

The bio-recognition mechanism of this sensor is based on the influence of the redox coupling reaction of the redox solution, such as a potassium ferrocyanide/potassium ferricyanide (K₃Fe(CN)₆/K₄Fe(CN)₆) solution, by the β-amyloid 42, T-Tau, and/or P-Tau and, respectively, an anti-β-amyloid 42, anti-T-Tau, and/or anti-P-Tau antibody. In the detection of β-amyloid 42, T-Tau, and/or P-Tau, the anti-β-amyloid 42 antibody, anti-T-Tau antibody, and/or anti-P-Tau antibody is used to provide a lock-and-key bio-recognition mechanism. The β-amyloid 42, T-Tau, and/or P-Tau interacts with, respectively, the anti-β-amyloid 42 antibody, anti-T-Tau antibody, and/or anti-P-Tau antibody affecting the electron charge transfer and can influence a redox coupling reaction in the redox solution applied to the working electrode. The level of β-amyloid 42, T-Tau, and/or P-Tau bound to the anti-β-amyloid 42, anti-T-Tau, and/or anti-P-Tau can be determined by measuring current flow between the working and counter electrode to which the sample and redox solution has been applied and comparing the measured current to control value, which can be based on a measured current between the working electrode and counter electrode that is free of bound anti-β-amyloid 42, anti-T-Tau, and/or anti-P-Tau.

Differential pulse voltammetry (DPV) can employed as the transduction mechanism of this biosensor to determine the level of bound anti-β-amyloid 42, anti-T-Tau, and/or anti-P-Tau. DPV applies a linear sweep voltammetry with a series of regular voltage pulses superimposed on the linear potential sweep. The current can then measured immediately before each potential change. Thus, the effect of the charging current could be minimized, achieving a higher sensitivity.

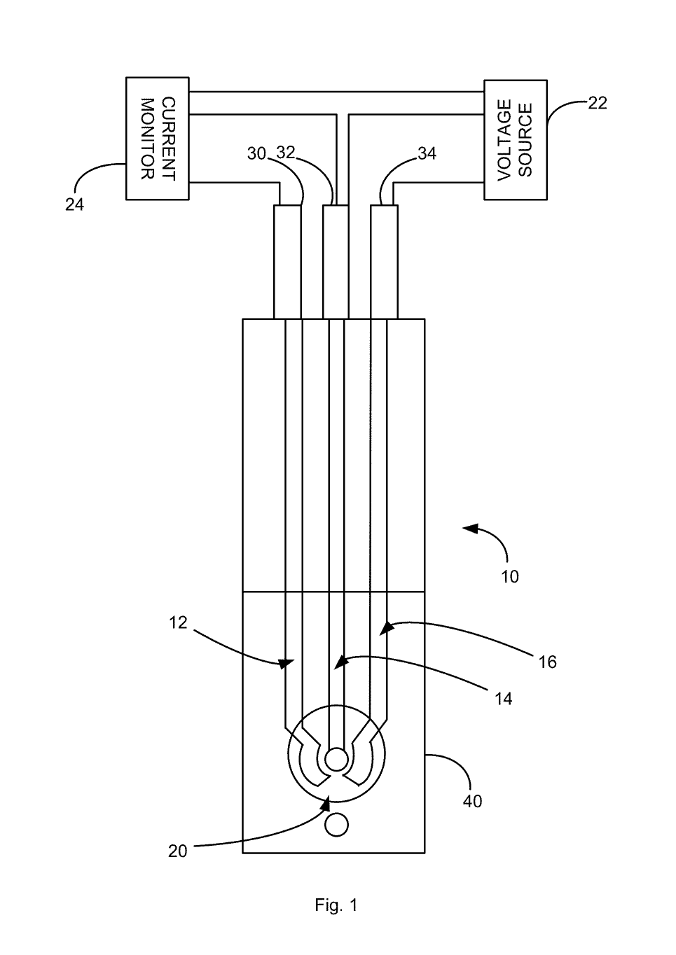

FIG. 1 illustrates a biosensor 10 of the system in accordance with an embodiment of the application. The sensor 10 is a three-electrode sensor including a counter electrode 12, a working electrode 14, and a reference electrode 16 that are formed on a surface of a substrate. A dielectric layer 40 covers a portion of the working electrode 12, counter electrode 14 and reference electrode 16. The dielectric layer 40 includes an aperture 20 that defines a detection region of the working electrode 12, counter electrode 14, and reference electrode 16, which is exposed to samples containing anti-β-amyloid 42, anti-T-Tau, and/or anti-P-Tau to be detected. An anti-β-amyloid 42 antibody, anti-T-Tau antibody, and/or anti-P-Tau antibody can be functionalized or chemically functionalized to the working electrode. The anti-β-amyloid 42 antibody, anti-T-Tau antibody, and/or anti-P-Tau antibody can bind selectively to, respectively, β-amyloid 42, T-Tau, and/or P-Tau in the biological sample.

The system further includes a measuring device that includes a voltage source 22 for applying a voltage potential to the working electrode, counter electrode, and/or reference electrode and a current monitor 24 for measuring the current flow between the working electrode and counter electrode.

The interaction of the anti-β-amyloid 42 antibody, anti-T-Tau antibody, and/or anti-P-Tau antibody and β-amyloid 42, T-Tau, and/or P-Tau in the presence of a redox solution can be detected using electrochemical analytical techniques, such as cyclic voltammetry (CV), differential pulse voltammetry (DPV), to determine the presence of the analyte in the sample. The working electrode 14 is poised at an appropriate electrochemical potential such that the current that flows through the electrode changes when the anti-β-amyloid 42 antibody, anti-T-Tau antibody, and/or anti-P-Tau antibody binds to β-amyloid 42, T-Tau, and/or P-Tau in the sample in the presence of the redox solution. The function of the counter electrode 12 is to complete the circuit, allowing charge to flow through the sensor 10.

The working electrode 14 and the counter electrode 12 are preferably formed of the same material, although this is not a requirement. Examples of materials that can be used for the working electrode 14 and counter electrode 12 include, but are not limited to, gold, platinum, palladium, silver, carbon, alloys thereof, and composites thereof.

The anti-β-amyloid 42, anti-T-Tau, and/or anti-P-Tau antibody, which is functionalized or chemically functionalized to the working electrode, can be an antibody that binds selectively to β-amyloid 42, T-Tau, and/or P-Tau. An antibody that binds selectively to β-amyloid 42, T-Tau, and/or P-Tau can be a monoclonal or polyclonal anti-β-amyloid 42 antibody, anti-T-Tau antibody, and/or anti-P-Tau antibody that binds selectively or specifically to, respectively, β-amyloid 42, T-Tau, and/or P-Tau. An anti-β-amyloid 42 antibody, anti-T-Tau antibody, and/or anti-P-Tau antibody having binding affinities in the picomolar to micromolar range are suitable. Such interaction can be reversible or irreversible.

The term “functionalized” or “chemically functionalized,” as used herein, means addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material so that it has desired biological function, which will he readily appreciated by a person of skill in the related art, such as bioengineering.

The anti-β-amyloid 42 antibody, anti-T-Tau antibody, and/or anti-P-Tau antibody may be functionalized to the working electrode covalently or non-covalently. Covalent attachment of an anti-β-amyloid 42 antibody, anti-T-Tau antibody, and/or anti-P-Tau antibody to the working electrode may be direct or indirect (e.g., through a linker). Anti-β-amyloid 42, anti-T-Tau, and/or anti-P-Tau antibodies may be immobilized on the working electrode using a linker. The linker can be a linker that can be used to link a variety of entities.

In some embodiments, the linker may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are N-hydroxysuccinimide (NHS), bis(sulfosuccinimidyl) suberate, bis[2-(succinimidooxycarbonyloxy)ethyl]sulfone, disuccinimidyl suberate, disuccinimidyl tartarate, N-succinimidyl S-acetylthioacetate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4[N-maleimidomethyl]-cyclohexane-1-carboxylhydrazide HCl, 4-(4-N-maleimidophenyl)-butyric acid hydrazide 2HCl, and 3[2-pyridyldithio]propionyl hydrazide.

Alternatively, anti-β-amyloid 42 antibodies, anti-T-Tau antibodies, and/or anti-P-Tau antibodies may be non-covalently coated onto the working electrode. Non-covalent deposition of the anti-β-amyloid 42 antibody, anti-T-Tau antibody, and/or anti-P-Tau antibody to the working electrode may involve the use of a polymer matrix. The polymer may be naturally occurring or non-naturally occurring and may be of any type including but not limited to nucleic acid (e.g., DNA, RNA, PNA, LNA, and the like, or mimics, derivatives, or combinations thereof), amino acids (e.g., peptides, proteins (native or denatured), and the like, or mimics, derivatives, or combinations thereof, lipids, polysaccharides, and functionalized block copolymers. The anti-β-amyloid 42 antibody, anti-T-Tau antibody, and/or anti-P-Tau antibody may be adsorbed onto and/or entrapped within the polymer matrix.

Alternatively, the anti-β-amyloid 42 antibody, anti-T-Tau antibody, and/or anti-P-Tau antibody may be covalently conjugated or crosslinked to the polymer (e.g., it may be “grafted” onto a functionalized polymer).

An example of a suitable peptide polymer is poly-lysine (e.g., poly-L-lysine). Examples of other polymers include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

In one particular embodiment, the working electrode can comprise a gold working electrode that is coated with a self-assembled monolayer (SAM) of 3-mercaptopropionic acid (MPA). The MPA molecule includes a thiol functional group at one end with an affinity for gold and a carboxylic group at the other end, which can covalently bond to proteins through a peptide bond after activation. The SAM of MPT can be activated by reaction with N-(3-dimethylaminopropyl)-N′-ethylcarbodiimide hydrochloride (EDC) and N-hydroxysuccinimide (NHS), which can further react with amine groups of proteins and antibodies.

In some embodiments, the anti-β-amyloid 42 antibody, anti-T-Tau antibody, and/or anti-P-Tau antibody can include monoclonal and polyclonal antibodies, immunologically active fragments (e.g., Fab or (Fab)2 fragments), antibody heavy chains, humanized antibodies, antibody light chains, and chimeric antibodies. Anti-β-amyloid 42 antibodies, anti-T-Tau antibodies, and/or anti-P-Tau antibodies, including monoclonal and polyclonal antibodies, fragments and chimeras, may be prepared using methods known in the art (see, for example, R. G. Mage and E. Lamoyi, in “Monoclonal Antibody Production Techniques and Applications”, 1987, Marcel Dekker, Inc.: New York, pp. 79-97; G. Kohler and C. Milstein, Nature, 1975, 256: 495-497; D. Kozbor et al., J. Immunol. Methods, 1985,81: 31-42; and R. J. Cote et al., Proc. Natl. Acad. Sci. 1983, 80: 2026-203; R. A. Lerner, Nature, 1982, 299: 593-596; A. C. Nairn et al., Nature, 1982,299: 734-736; A. J. Czernik et al., Methods Enzymol. 1991, 201: 264-283; A. J. Czernik et al., Neuromethods: Regulatory Protein Modification: Techniques & Protocols, 1997, 30: 219-250; A. J. Czemik et al., NeuroNeuroprotocols, 1995, 6: 56-61; H. Zhang et al., J. Biol. Chern. 2002, 277: 39379-39387; S. L. Morrison et al., Proc. Natl. Acad. Sci., 1984, 81: 6851-6855; M. S. Neuberger et al., Nature, 1984,312: 604-608; S. Takeda et al., Nature, 1985, 314: 452-454). Antibodies to be used in the biosensor can be purified by methods well known in the art (see, for example, S. A. Minden, “Monoclonal Antibody Purification”, 1996, IBC Biomedical Library Series: Southbridge, Mass.). For example, anti-β-amyloid 42 antibodies, anti-T-Tau antibodies, and/or anti-P-Tau antibodies can be affinity purified by passage over a column to which a protein marker or fragment thereof is bound. The bound antibodies can then be eluted from the column using a buffer with a high salt concentration.

Instead of being prepared, anti-β-amyloid 42 antibodies, anti-T-Tau antibodies, and/or anti-P-Tau antibodies to be used in the methods described herein may be obtained from scientific or commercial sources.

In order to minimize any non-specific binding on the working electrode surface and blocking any open surface area of the working electrode at least one blocking agent can be applied to the surface of the working electrode once the anti-β-amyloid 42 antibody, anti-T-Tau antibody, and/or anti-P-Tau antibody has been functionalized or chemically functionalized to the working electrode. The blocking agent can enhance the reproducibility and sensitivity of the biosensor by minimizing non-specific interactions on the working electrode. In some embodiments, the blocking agent can include dithiothreitol or casein. The blocking agent can be applied to the surface of the working at an amount effective to minimize non-specific binding of proteins or other molecules on the surface of the working electrode.

The redox solution is applied to the working electrode for determining the quantity of β-amyloid 42, T-Tau, and/or P-Tau in the sample bound to the anti-β-amyloid 42, anti-T-Tau, and/or anti-P-Tau antibody. The redox coupling solution can include a redox mediator, such as potassium ferrocyanide/potassium ferricyanide (K₃Fe(CN)₆/K₄Fe(CN)₆), that is provided at equimolar concentration in a PBS solution.

The voltage source 22 can apply a voltage potential to the working electrode 14 and reference and/or counter electrode 16, 12, depending on the design of the sensor 10. The current between the working electrode 14 and counter electrode 16 can be measured with the measuring device or meter 24. Such current is dependent on interaction of 17 β-estradiol in the sample with the anti-β-amyloid 42 antibodies, anti-T-Tau antibodies, and/or anti-P-Tau antibodies on the working electrode.

The amount or level of current measured is proportional to the level or amount of β-amyloid 42, T-Tau, and/or P-Tau in the sample. In some embodiments, where the sample is a blood or cerebrospinal fluid, once the current level generated by the sample and redox solution tested with the sensor is determined, the level can be compared to a predetermined value or control value to provide information for monitoring the presence or absence of β-amyloid 42, T-Tau, and/or P-Tau in the bodily sample.

In other embodiments, where the sample is a bodily sample obtained from a subject, once the current level generated by the reaction solution tested with the sensor is determined, the level can be compared to a predetermined value or control value to provide information for diagnosing or monitoring of the condition, pathology, or disorder in a subject that is associated with presence or absence of β-amyloid 42, T-Tau, and/or P-Tau.

The current level generated by sample obtained from the subject can be compared to a current level of a sample previously obtained from the subject, such as prior to administration of a therapeutic. Accordingly, the methods described herein can be used to measure the efficacy of a therapeutic regimen for the treatment of a condition, pathology, or disorder associated with the level of the β-amyloid 42, T-Tau, and/or P-Tau in a subject by comparing the current level obtained before and after a therapeutic regimen. Additionally, the methods described herein can be used to measure the progression of a condition, pathology, or disorder associated with the presence or absence of the β-amyloid 42, T-Tau, and/or P-Tau in a subject by comparing the current level in a bodily sample obtained over a given time period, such as days, weeks, months, or years.

The current level generated by a sample obtained from a subject may also be compared to a predetermined value or control value to provide information for determining the severity or aggressiveness of a condition, pathology, or disorder associated with β-amyloid 42, T-Tau, and/or P-Tau levels in the subject. A predetermined value or control value can be based upon the current level in comparable samples obtained from a healthy or normal subject or the general population or from a select population of control subjects.

The predetermined value can take a variety of forms. The predetermined value can be a single cut-off value, such as a median or mean. The predetermined value can be established based upon comparative groups such as where the current level in one defined group is double the current level in another defined group. The predetermined value can be a range, for example, where the general subject population is divided equally (or unequally) into groups, or into quadrants, the lowest quadrant being subjects with the lowest current level, the highest quadrant being individuals with the highest current level. In an exemplary embodiment, two cutoff values are selected to minimize the rate of false positive and negative results.

The biosensor illustrated in FIG. 1 can be fabricated on a substrate 100 formed from polyester or other electrically non-conductive material, such as other polymeric materials, alumina (Al₂O₃), ceramic based materials, glass or a semi-conductive substrate, such as silicon, silicon oxide and other covered substrates. Multiple sensor devices can thus be formed on a common substrate. As will be appreciated, variations in the geometry and size of the electrodes are contemplated.

The biosensor can be made using a thin film, thick film, and/or ink-jet printing technique, especially for the deposition of multiple electrodes on a substrate. The thin film process can include physical or chemical vapor deposition. Electrochemical sensors and thick film techniques for their fabrication are discussed in U.S. Pat. No. 4,571,292 to C. C. Liu et al., U.S. Pat. No. 4,655,880 to C. C. Liu, and co-pending application U.S. Ser. No. 09/466,865, which are incorporated by reference in their entirety.

In some embodiments, the working electrode, counter electrode, and reference electrode may be formed using laser ablation, a process which can produce elements with features that are less than one-thousandth of an inch. Laser ablation enables the precise definition of the working electrode, counter electrode, and reference electrode as well as electrical connecting leads and other features, which is required to reduce coefficient of variation and provide accurate measurements. Metalized films, such as Au, Pd, and Pt or any metal having similar electrochemical properties, that can be sputtered or coated on plastic substrates, such as PET or polycarbonate, or other dielectric material, can be irradiated using laser ablation to provide these features.

In one example, a gold film with a thickness of about 300 A to about 2000 A can be deposited by a sputtering technique resulting in very uniform layer that can be laser ablated to form the working and counter electrodes. The counter electrode can use other materials. However, for the simplicity of fabrication, using identical material for both working and counter electrodes will simplify the fabrication process providing the feasibility of producing both electrodes in a single processing step. An Ag/AgCl reference electrode, the insulation layer, and the electrical connecting parts can then be printed using thick-film screen printing techniques.

The working electrode surface can then be cross-linked or biotinylated chemically in order to allow the attachment of an anti-β-amyloid 42 antibody, anti-T-Tau antibody, and/or anti-P-Tau antibody. The crosslinking step can be accomplished by generating thiol bonds. This can be chemically accomplished using, for example, a self-assembled monolayer (SAM) of 3-mercaptopropionic acid (MPA). The MPA molecule includes a thiol functional group at one end with an affinity for gold and a carboxylic group at the other end, which can covalently bond to proteins through peptide bond after activation. The SAM of MPT can be activated for binding to a protein, such as an antibody, by reaction with N-(3-dimethylaminopropyl)-N′-ethylcarbodiimide hydrochloride (EDC) and N-hydroxysuccinimide (NHS) that can further react with amine groups of proteins and antibodies. Similar chemical methods can be used to produce semi-stable amine-ester groups to enhance the cross linking between the antibodies and the thiol groups. Other cross-linking agent, such as 3,3′-dithiobis[sulfosuccinimidylpropionate] (DTSSP), can also be used in this process.

Biotinylation is rapid, specific and is normally unperturb to the natural function of the molecule due to the relatively small size of biotin. Streptavidin and similar chemicals such as avidin can be immobilized on the working electrode surface for a biosensor for the detection of an interaction of anti-β-amyloid 42 antibody, anti-T-Tau antibody, and/or anti-P-Tau antibody and anti-β-amyloid 42, anti-T-Tau, and/or anti-P-Tau.

Following addition of anti-3-amyloid 42 antibody, anti-T-Tau antibody, and/or anti-P-Tau antibody to the working electrode, the working electrode surface can be blocked using a blocking agent to minimize any non-specific molecule (e.g., protein) bonding on the electrode surface. This step will enhance the reproducibility and sensitivity of the biosensor. In some embodiments, DTT (Dithiothreitol), casein, and/or other blocking agents can be used to cover the open surface area of the working electrode and minimize any non-specific protein coverage.

In other embodiments, a plurality of biosensors can be provided on a surface of a substrate to provide a biosensor array. The biosensor array can be configured to detect β-amyloid 42, T-Tau, and/or P-Tau concentration changes in a host of chemical and/or biological processes occurring in proximity to the array. The biosensor array can include a plurality biosensors arranged in a plurality of rows and a plurality of columns. Each biosensor can use a working electrode, a counter electrode, and a dielectric layer covering a portion of the working electrode and counter electrode and defining an aperture exposing other portions of the working electrode and counter electrode. Anti-β-amyloid 42, anti-T-Tau, and/or anti-P-Tau antibodies for β-amyloid 42, T-Tau, and/or P-Tau can be functionalized or chemically functionalized to the working electrode. The anti-3-amyloid 42, anti-T-Tau, and/or anti-P-Tau antibodies can be the same or different for each biosensor of the array and can bind selectively to β-amyloid 42, T-Tau, and/or P-Tau. The biosensors of the array can be configured to provide at least one output signal representing the presence and/or concentration of β-amyloid 42, T-Tau, and/or P-Tau proximate to a surface of the array. For each column of the plurality of columns or for each row of the plurality of rows, the array further comprises column or row circuitry configured to provide voltage potentials to respective biosensors in the column or row. Each biosensor in the row or column can potentially detect a different analyte and/or biased to detect different analytes.

### Example 1

The development of a cost-effective, single-use, disposable in vitro biosensor system which can measure T-Tau in blood accurately can be a first step in providing a practical and useful tool in the assessment of neurodegenerative disorders. In this example, we describe a biosensor for bio-recognition of T-Tau and β-amyloid 42. The bio-recognition mechanism of this biosensor system is based on the antibody and antigen interaction and the effect of a [Fe(CN)6]3-/4-redox probe by this interaction. The transduction mechanism is the electrochemical differential pulse voltammetry (DPV) technique. The biosensor was manufactured using a cost-effective roll-to-roll process. Both the working and the counter electrodes were thin gold films, and the reference electrode was a thick-film-printed Ag/AgCl electrode. The Thiol based chemical functionalization step was used to link the antibody to the gold electrode surface. Activation of the carboxylic group on one end of the MPA molecule for the immobilization of anti-T-Tau (antibody of T-Tau) was accomplished by the carbodiimide conjugation technique and showed excellent MPA surface coverage of the biosensor by X-ray photoelectron spectroscopy (XPS) characterization. DPV measurements of T-Tau antigen in both 0.1 M PBS and undiluted human serum showed excellent results. Therefore, the results of this study suggested that a useful tool for the single-use, disposable in vitro measurement of T-Tau to assess neuro-degenerative disorders including Alzheimer's disease, TBI, and other dementia symptoms in blood serum was feasible.

Diagnosis of T-Tau in one of the neuro-degenerative disorders, Creutzfeldt-Jacob disease, in cerebrospinal fluid used a 1400 pg/mL as the cutoff level of T-Tau in CSF based on Swedish Mortality Registry. Thus, the level of 1400 pg/mL of T-Tau in a physiological fluid is used as the guide for identifying neuro-degenerative disorders.

### Materials and Methods

### Apparatus and Reagents

Tau protein, 6 isoforms (Cat. No. T7951), and anti-T-Tau (Cat. No. SAB 5500182) of rabbit monoclonal antibody were both obtained from Sigma Aldrich (St. Louis, Mo., USA). Phosphate-buffer saline (PBS) 1.0 M (pH 7.4), human serum, 3-mercaptopropionic acid (MPA), N-(3 dimethylaminopropyl)-N′-ethylcarbodiimide hydrochloride (EDC), and N-hydroxysuccinimide (NHS) were also purchased from Sigma-Aldrich (St. Louis, Mo., USA). Potassium hydroxide pellets, concentrated H₂SO0₄ (95.0 to 98.0 w/w %), and concentrated HNO₃ (70% w/w %) were obtained from Fisher Scientific (Pittsburgh, Pa.). Recombined human beta-amyloid 42 was used in the interference study (Cat. No. ab82795) ABCAM, (Cambridge, Mass., USA). All chemicals were used without further purification. A CHI 660C (CH Instrument, Inc., Austin, Tex., USA) Electrochemical Workstation was used for DPV and EIS investigations. All experiments were conducted at room temperature. X-ray photoelectron spectroscopy (XPS) was performed by a PHI Versaprobe 5000 Scanning X-Ray Photoelectron Spectrometer.

### Design of the Biosensor

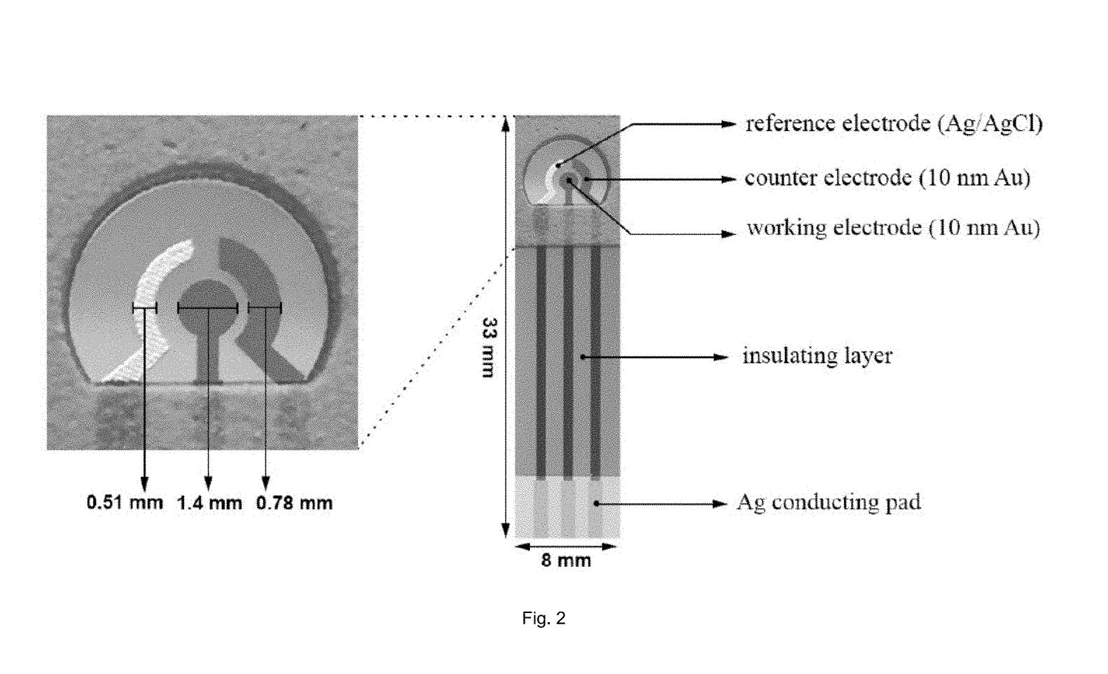

This biosensor prototype for the detection of T-Tau has a three-electrode configuration. Both the working and counter electrodes were thin gold films 10 nm in thickness. The gold film was deposited by sputtering physical vapor deposition at an atomic level. No binder was employed in any thick-film-printed biosensor including commercially available three-electrode based biosensors. Therefore, the surface of the gold film working electrode was uniform and reproducible. The reference electrode was a thick-film-printed Ag/AgCl electrode. The whole biosensor was formed on a polyethylene terephalate (PET) substrate. Laser ablation technique was used to define the size and dimensions of the biosensor and its electrode elements. The insulator was a thick-film-printed silicon-free dielectric layer made of Nazdar APL 34 ink (Shawnee, Kans., USA). This was a cost-effective roll-to-roll fabrication process. In this study, 100 individual biosensors in 4 rows were produced on each PET substrate (355 280 mm2). FIG. 2 shows the structure and actual dimensions of this biosensor. The overall dimensions of an individual biosensor were 33.0 8.0 mm². The working electrode area was 1.54 mm2 accommodating 20-25 μL of liquid test sample required in this study. A more detailed description of the design and fabrication process of this biosensor has been presented elsewhere.

### Pretreatment of the Biosensor

Result reproducibility of the biosensor prototype is important for any accurate measurement of the analyte (Tau antigen in this study). Thus, a pretreatment experimental protocol intended to enhance the reproducibility of this thin gold film-based biosensor was established based on previous reports. Since the gold film electrodes used in this study were relatively thin, and in order to maintain the integrity of the biosensor prototype, modifications of the pretreatment procedure were needed in comparison to the bulk gold nanoparticles or to the treated particles. This cleaning process would decrease the electrode charge transfer resistance, thus improving the sensitivity of the biosensor.

In a typical pretreatment procedure, a row of 5 or 7 biosensors was immersed in a 2 M KOH solution for 15 min. After rinsing with copious amount of DI water, the biosensors were placed in a 20-fold diluted concentrated H₂SO₄ solution (95.0 to 98.0 w/w %) for another 15 min. DI water was then used to rinse the biosensor prototypes. The biosensors were then placed in a 20-fold diluted concentrated HNO₃ solution (70% w/w %) for another 15 min. The biosensors were rinsed once more and then dried by a gentle flow of nitrogen gas. This pretreatment procedure resulted in a significant decrease in electrode charge transfer resistance, enhancing the sensitivity and reproducibility of the biosensor.

### Characterization of the Surface Area

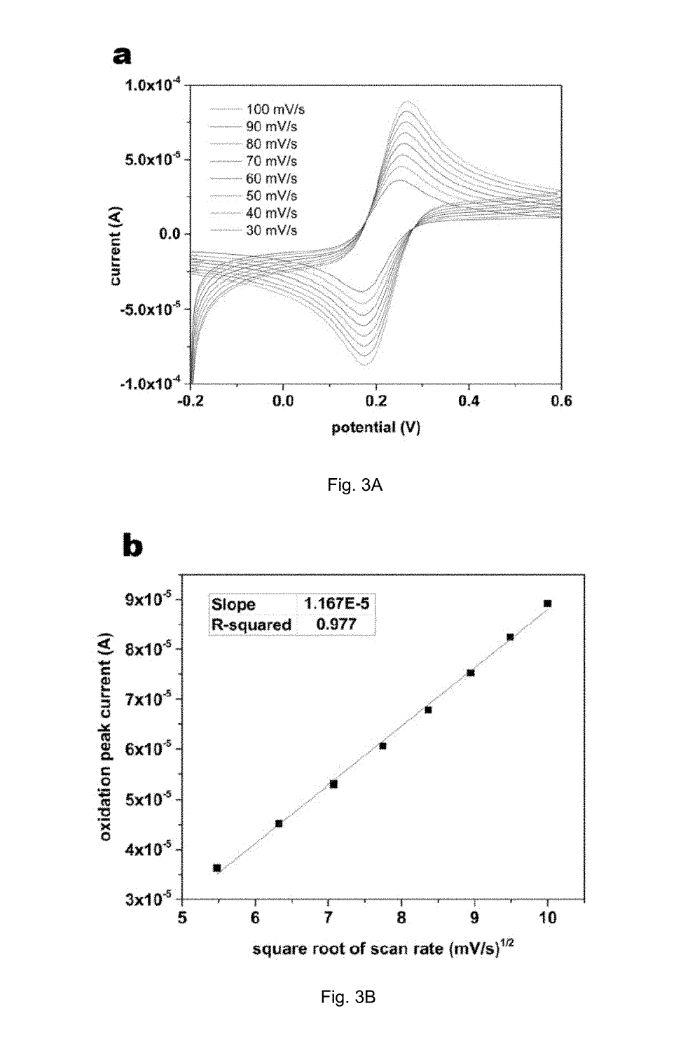

Responses of biosensors were characterized by assessing the stability and reproducibility of the electrochemical surface area. Cyclic voltammograms were obtained at scan rates ranging from 30 to 100 mV/s in a solution of K₃Fe(CN)₆ and K₄Fe(CN)₆, 5 mM in each component, having 0.1 M KCl (FIG. 3A). As presented in FIG. 3B, oxidation peak current presented a linear relationship versus the square root of the scan rate. Assuming the diffusion coefficient of ferricyanide ion to be constant, this linear relationship demonstrated the stability of electro-active surface area based on the Randles-Sevcik equation. Using the equation, the calculated electro-active surface area showed less than 2% relative standard deviation from sensor to sensor (n=3), signifying high reproducibility.

Immobilization of T-Tau Antibody onto the Gold Working Electrode of the Biosensor

In a typical experiment, 5 to 7 biosensors were subjected to surface modification in a single batch. SAM of MPA was employed to covalently immobilize anti-T-Tau on the surface of the gold electrode. The MPA molecule consisted of a thiol functional group at one end, which processed a great affinity to gold, and a carboxylic group at another end which was suitable for bonding covalently to proteins through a peptide bond after an activation procedure. Thiol modification of the gold electrode surface for protein immobilization was a well-developed technique. The biosensors were immersed in 1 mM solution of MPA in pure ethanol for 24 h, rinsed with DI water, and dried in a steam of N₂. The MPA-modified biosensors were incubated in 0.1 M PBS (pH=7.4) containing 0.25 M EDC and 0.05 M NHS for 5 h to activate MPA carboxylic groups. The activated biosensors were then rinsed by 0.1 M PBS and dried by N2 flow. 5 μL of 0.05 mg/mL anti-T-Tau was then casted on the sensing area of each biosensor and left to dry overnight at 4° C. Antibody-immobilized biosensors were rinsed with 0.1 M PBS to remove loosely bonded proteins. The biosensors were then dried again under a steam of N2 and stored at 4° C. This immobilization process was very similar to one described elsewhere. XPS was used to characterize the MPA-SAM coverage of the gold working electrode (results not shown). Identical to our previous study, the surface coverage of MPA-SAM was high and the formation of Au—S covalent bond together with the upward orientation of MPA-SAM carboxylic groups, were confirmed. This showed that the immobilization procedure was effective. The XPS results were in agreement with those reported by others. Therefore, the anti-T-Tau bonded biosensor was ready for T-Tau antigen detection.

### Differential Pulse Voltammetry (DPV) Measurement

DPV is a well-established electroanalytical technique; however, its applications to biomedical measurement has not been fully exploited. Cyclic voltammetry (CV) and chronoamperometry (CA) are generally used in biomedical measurements. Both CV and CA provide sufficient sensitivity in practical biomedical applications. The required electronic interface for CV and CA are relatively simple. However, DPV applies a series of regular potential pulse superimposed on the potential stair steps. The current is then measured immediately prior to each potential change. Consequently, the charging current can be minimized, resulting in a higher sensitivity. It is based on this technical advantage that DPV was used for the detection of T-Tau using our biosensor system. The anti-T-Tau was first bonded and functionalized as described above. The biosensors were then incubated in solutions of T-Tau with different concentrations for 3 h at room temperature. Antigen solutions were prepared both in 0.1 M PBS and undiluted human serum. After the incubation, biosensors were rinsed with 0.1M PBS removing any unbonded T-Tau. A solution of K₃Fe(CN)₆ and K₄Fe(CN)₆, 5 mM in each component, was prepared in 0.1 M PBS, 20 μL of this redox probe was dropped to the sensing area of the biosensor, and the DPV measurement then took place.

In this study, an Electrochemical Workstation (CHI 660 model C) was used. The software of the Workstation provided direct experimental setting for the DPV measurement. In a typical measurement, the initial potential was set at −0.3V and the final potential was set at +0.3 V. The potential increase was set at 0.004 V, the amplitude at 0.05 V, and the pulse width at 0.05 s. The pulse period was set at 0.2 s.

### Results and Discussion

### Evaluation of the Pretreatment Procedure by Electrochemical Impedance Spectroscopy (EIS)

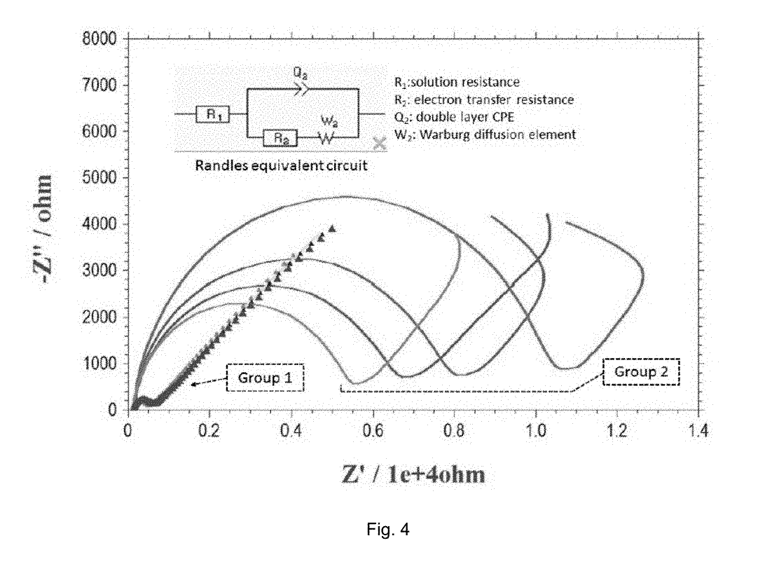

In order to validate the enhancement of the sensor response, reproducibility, and electron charge transfer due to the cleaning procedure, electrochemical impedance spectroscopy (EIS) was employed for two groups of sensors consisting of four sensors each. Sensors in Group 1 were subjected to the cleaning protocol described above, whereas sensors in Group 2 were cleaned by ethanol and deionized water (DIW) sequentially. A solution of K₃Fe(CN)₆ and K₄Fe(CN)₆, 5 mM of each component, was prepared in 0.1 M PBS and used for EIS tests. Twenty microliters of redox couple solution was casted on the sensing area of each sensor for EIS. FIG. 4 presents the EIS results obtained for the two groups of sensors in the form of a Nyquist plot using a frequency range of 10-2 to 104 Hz with 5 mV voltage amplitude. An equivalent electrical circuit model was fitted to EIS data using EC-lab software. Randles equivalent circuit was selected to model the experimental data. In a typical EIS measurement, the initial potential was set at 0.0 V. The high frequency was set at 10,000 Hz and the low frequency was set at 0.01 Hz. The amplitude was set at 0.005 V and the quiet time was set at 2 s.

Considering the physical structure of the interface, each component in the Randles circuit represents an element in the actual electrode/analyte physical interface. The semicircular region of the Nyquist plots associated with the electron transfer processes was modeled by a parallel circuit representation of a resistor (R₂) and the constant phase element (CPE). The tail at the lower frequencies indicated the presence of diffusion limited electrochemical processes, represented using the Warburg element (W₂). The solution resistance was represented by R1. Table 1 presents the calculated R2 values from Randles model data fitting for all the sensors tested. According to Table 1, the charge transfer resistance (R₂) that characterizes the interfacial electron transfer resulting from the K₃Fe(CN)₆/K₄Fe(CN)₆ redox couple, decreases significantly after the cleaning process when comparing the Group 2 sensors to the Group 1 sensors. Moreover, the data scattering, which could be observed for the electrodes in Group 2, was minimized by the cleaning procedure used for the Group 1 sensors. Thus, the EIS test successfully validated the profound effect of this cleaning procedure, demonstrating the excellent reproducibility of the sensors and the decrease in sensor charge transfer resistance.

TABLE 1
Calculated R2 (charge transfer resistance) values from
Randles model data fitting

| | Sensor #1 | Sensor | Sensor | Sensor #4 |
|---|---|---|---|---|
| Group 1 | 198 Ω | 200 Ω | 201 Ω | 204 Ω |
| Group 2 | 6150 Ω | 6913 Ω | 7941 Ω | 11346 Ω |

### Measurement of T-Tau Proteins in PBS Solution

The Tau protein ladder, human recombinant (Cat. No. T7951, Sigma Aldrich, St. Louis, Mo., USA) was used in this study. Adult brain Tau proteins are varied in size from 352 to 441 amino acids (approximately 36.8 to 45.9 kDa). This protein ladder contained 6 recombinant Tau proteins with molecular masses of 36.8, 39.7, 40.0, 42.6, 42.9, and 45.9 kDa, respectively. In the purchased T-Tau protein ladder, 50 μL contained 0.25 μg of each of the six isoforms. In this study, we did not intend to measure each isoform separately and only assessed that the parameter was the total T-Tau protein in the test medium. We used 0.1 M PBS as test medium (pH=7.4), and dissolved the T-Tau protein ladder in PBS over the range of 1000 pg/mL to 100,000 pg/mL. The anti-T-Tau concentration used was 500,000 pg/mL. This higher antibody concentration was used in order to minimize the possibility of its becoming a rate-limited component in this bio-recognition mechanism. It was feasible to modify and optimize this antibody concentration. This future investigation is beyond the scope of this presentation.

In order to explain the detection mechanism of the T-Tau biosensor, EIS measurements were carried out for biosensors incubated in solutions with different concentrations of T-Tau protein. FIG. 4 presents the EIS Nyquist plots obtained in presence of K₃Fe(CN)₆/K₄Fe(CN)₆ redox couple for antibody immobilized biosensors incubated in T-Tau solutions of 1000 and 100,000 pg/mL. Antigen solutions were prepared in 0.1 M PBS. A Randles equivalent circuit was fitted to the experimental data using EC-lab software. The data obtained from the circuit fitting is presented in Table 2. Ret value in Randles equivalent circuit is defined as the resistance to charge transfer of the electrochemical interface.

TABLE 2
Data obtained from Randles equivalent circuit modeling of EIS
Nyquist plots in FIG. 4

| T-Tau (pg/mL) | Q(μF) | Z_{w} (Ω) | Rₑₜ(Ω) | Rₛ(Ω) |
|---|---|---|---|---|
| 1000 | 1.33 | 1480 | 1154 | 125 |
| 100,000 | 120.8 | 1865 | 201.2 | 68.8 |

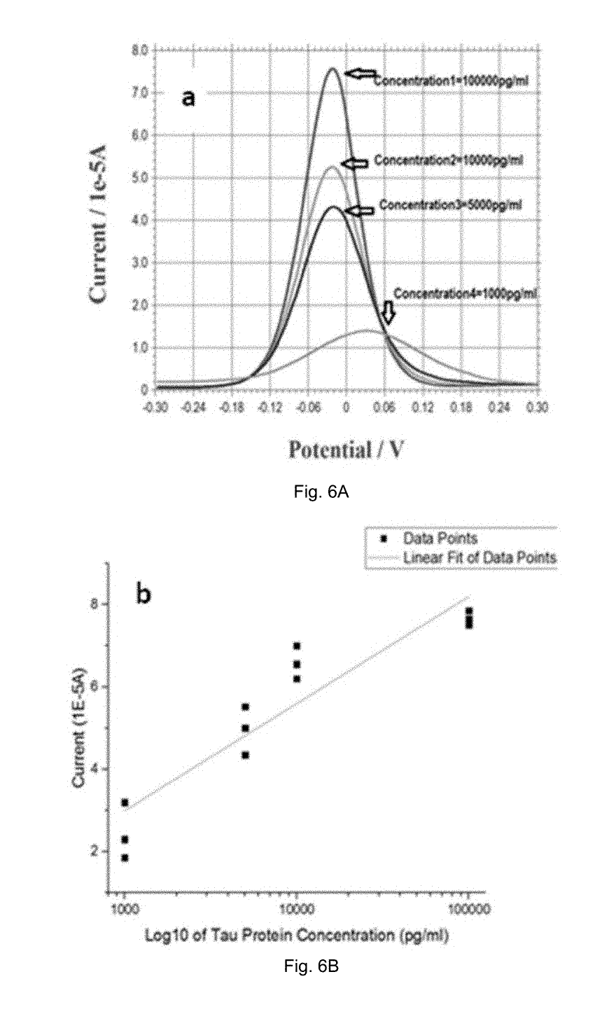

According to Table 2, resistance to charge transfer (Ret) of the sensing interface decreased significantly from 1154Ω for the sensor incubated in a T-Tau solution of 1000 pg/mL to 201.2Ω or the one incubated in a T-Tau solution of 100,000 pg/mL. As reported previously, the formation of T-Tau protein film on the surface leads to the development of positive charges, which enhance the charge permeability of the electrode to the negatively charged redox probe of [Fe(CN)₆]^{3-/4-}. Therefore, binding more Tau protein to the surface dramatically decreased the electrode resistance to the charge transfer of [Fe(CN)₆]^{3-/4-}. This phenomenon was exploited as the sensing mechanism in DPV measurements. FIG. 6 shows the DPV measurement results of T-Tau proteins in a 0.1 M PBS solution test medium as well as its calibration curve. According to the figure, the anodic peak current associated with one electron transfer reaction of [Fe(CN)₆]⁴⁻ to [Fe(CN)₆]³⁻ was increased by increasing the concentration of T-Tau.

FIG. 6A shows the typical DPV measurements of a T-Tau protein concentration of 1000 pg/mL to 100,000 pg/mL. Each measurement was accomplished using a single-use, disposable biosensor. The current outputs obtained were free-of-noise, as demonstrated in FIG. 6A. FIG. 6B is the calibration curve based on the DPV measurements of multiple experimental runs (n>3). The axis for the T-Tau protein concentration is in logarithmic scale, covering a wide range of T-Tau concentrations. The calibration curve is a linear least-square-fitting based on experimental data. A linear relationship of Y=2.6×4.7 was obtained, where Y is the current output of the biosensor and X is the T-Tau protein concentration. The R² value of this linear fitting is 0.85. One must recognize that this detection covered a very large concentration range of T-Tau protein and the modification step of the biosensor was carried out individually and manually. Thus, the uniformity of each biosensor was not 100% identical. Refinement could further enhance this linear relationship between the current outputs of the biosensor and the T-Tau concentration.

### Measurement of T-Tau Proteins in Undiluted Human Serum

DPV measurements for different T-Tau protein concentrations in undiluted human serum were also carried out in this study. Human serum (Cat No.3667, Sigma Aldrich, St. Louis, Mo., USA) was used to prepare antigen solutions.

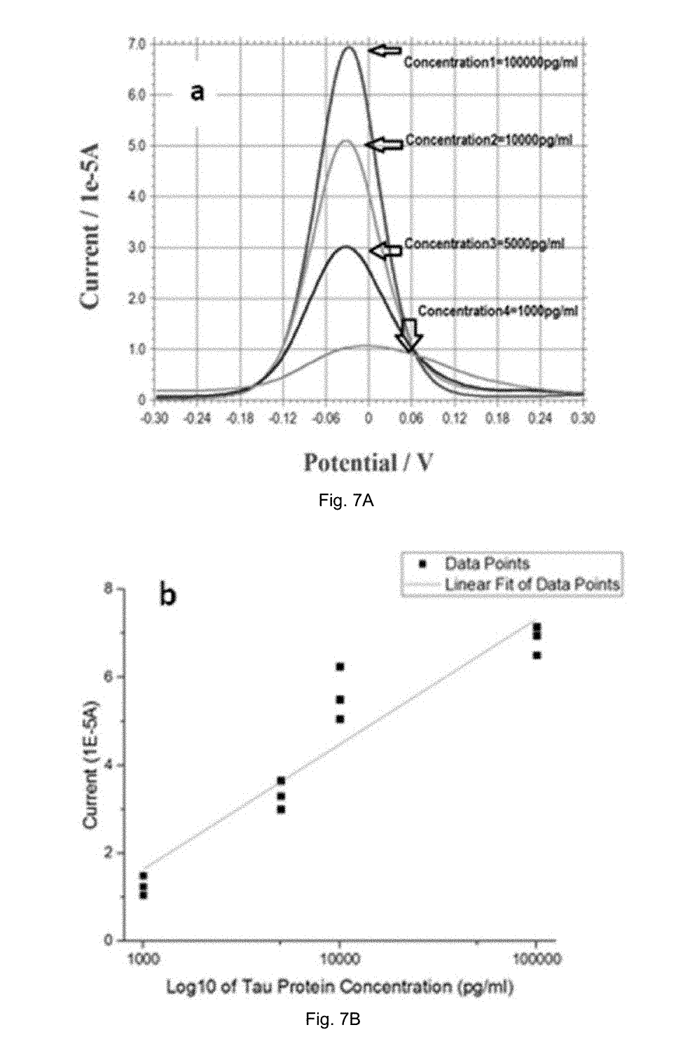

FIG. 7A shows the typical DPV measurements of various T-Tau protein concentrations in the serum. The T-Tau protein concentration was tested between 1000 pg/mL and 100,000 pg/mL. Similar to the test in 0.1 M PBS, a single-use, disposable biosensor was used for each measurement. FIG. 7B shows the corresponding calibration curve of DPV measurements in serum based on results shown in FIG. 7A with n=3. The anti-T-Tau concentration used in this phase of study was 500,000 pg/mL. FIG. 7B yields a least-square fitted calibration equation of Y=2.8×6.9, where Y is the current outputs of the biosensor and X is the T-Tau concentration in blood serum. The R₂ value of this equation was 0.88. It is feasible to optimize operational parameters further in this T-Tau detection system, such as the antibody concentration, the range of the detecting T-Tau proteins, concentration, and others. Our purpose of this presentation is to demonstrate that the basic designed biosensor with DPV transduction mechanism can be effectively used for T-Tau protein detection.

### Interference Study of T-Tau Proteins Measurement of This Biosensor

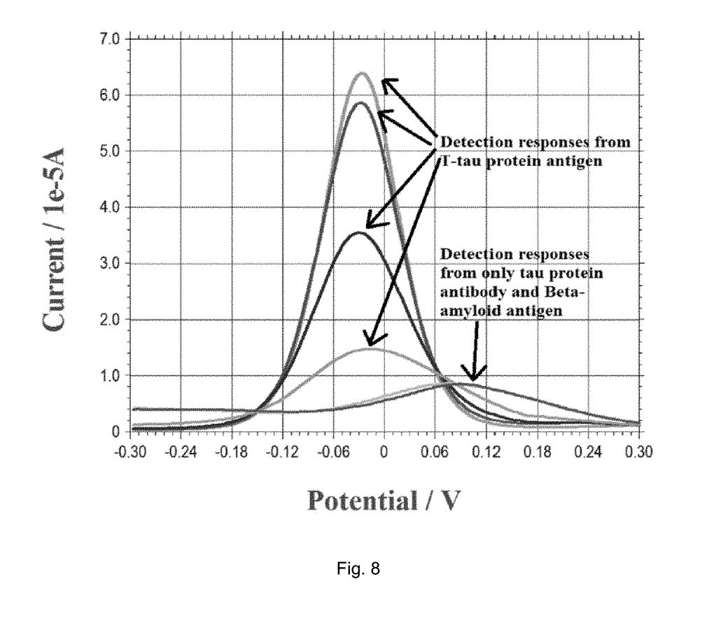

The bio-recognition mechanism of this biosensor was based on the interaction of the antibody and antigen of T-Tau protein, and this detection mechanism was very specific. However, in order to examine any potential interference, β-amyloid 42, another important biomarker of neuro-degenerative disorders, was used in this interference study. The T-Tau detection biosensor was prepared as described previously. The anti-T-Tau concentration used was maintained at 500,000 pg/mL Recombined human β-amyloid 42 at a concentration of 50,000 pg/mL with an incubation time of 2 h, identical to a previous study in PBS, was then used in this study. FIG. 8 shows the testing results. Both the β-amyloid 42 antigen and the zero concentration of T-Tau PBS solution show the same base line as comparing to other T-Tau protein-contained PBS solutions as presented in FIG. 6. This study confirmed positively that this T-Tau biosensor was specific to T-Tau protein only.

A cost-effective single-use, in vitro biosensor for the detection of a biomarker of neuro-degenerative disorder, T-Tau protein, has been designed, manufactured, and evaluated in phosphate-buffer saline and undiluted human serum. DPV was used as the measurement technique. Measurements of T-Tau protein in both 0.1 M PBS and undiluted human serum over the concentration range of 1000 pg/mL to 100,000 pg/mL showed excellent results and good linearity of the calibration curves. This biosensor platform technology can be further optimized and can be applied to detect other biomarkers of neuro-degenerative disorders, including P-Tau protein and β-amyloid 42.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention the following is claimed:

1. A detection system for detecting β-amyloid 42, T-Tau, and/or P-Tau levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-β-amyloid 42 antibody, anti-T-Tau antibody, and/or anti-P-Tau antibody functionalized or chemically functionalized to a surface of the exposed portion of the working electrode, the anti-β-amyloid 42 antibody, anti-T-Tau antibody, and/or anti-P-Tau antibody selectively binding to, respectively, β-amyloid 42, T-Tau, and/or P-Tau in a sample and the β-amyloid 42, T-Tau, and/or P-Tau once bound being detectable by measuring the current flow between the working electrode and counter electrode,
a redox solution that is applied to the working electrode for determining the quantity of β-amyloid 42, T-Tau, and/or P-Tau in the sample bound, respectively, to the anti-β-amyloid 42, anti-T-Tau, and/or anti-P-Tau antibody, and
a measuring device for applying voltage potentials to the working electrode and counter electrode and measuring the current flow between the working electrode and counter electrode.

2. The system of claim 1, wherein the working electrode and the counter electrode comprise metalized films.

3. The system of claim 2, wherein the working electrode and counter electrode independently comprise gold, platinum, palladium, silver, carbon, alloys thereof, and composites thereof.

4. The system of claim 2, wherein the metalized films are provided on the surface of the substrate by sputtering or coating the films on the surface and wherein the working electrode and the counter electrode are formed using laser ablation to define the dimensions of the working electrode and the counter electrode.

5. The system of claim 1, wherein the redox solution comprises potassium ferrocyanide/potassium ferricyanide solution.

6. The system of claim 1, further comprising a reference electrode on the surface of the substrate, the dielectric covering a portion of the reference electrode.

7. The system of claim 1, the anti-β-amyloid 42 antibody, anti-T-Tau antibody, and/or anti-P-Tau antibody being chemically functionalized to the surface of the working electrode coated with a 3-mercaptopropionic acid (MPA) monolayer.

8. The system of claim 1, wherein the sample comprises blood or serum.

9. A detection system for detecting β-amyloid 42, T-Tau, and/or P-Tau levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-β-amyloid 42 antibody, anti-T-Tau antibody, and/or anti-P-Tau antibody functionalized or chemically functionalized to a surface of the exposed portion of the working electrode, the anti-β-amyloid 42 antibody, anti-T-Tau antibody, and/or anti-P-Tau antibody selectively binding to, respectively, β-amyloid 42, T-Tau, and/or P-Tau in a sample and the β-amyloid 42, T-Tau, and/or P-Tau once bound being detectable by measuring the current flow between the working electrode and counter electrode, an equimolar potassium ferrocyanide/potassium ferricyanide redox solution that is applied to the working electrode for determining the quantity of β-amyloid 42, T-Tau, and/or P-Tau in the sample bound to, respectively, the anti-β-amyloid 42, anti-T-Tau, and/or anti-P-Tau antibody, and
a measuring device for applying voltage potentials to the working electrode and counter electrode and measuring the current flow between the working electrode and counter electrode.

10. The system of claim 9, wherein the working electrode and the counter electrode comprise metalized films.

11. The system of claim 9, wherein the working electrode and counter electrode independently comprise gold, platinum, palladium, silver, carbon, alloys thereof, and composites thereof.

12. The system of claim 10, wherein the metalized films are provided on the surface of the substrate by sputtering or coating the films on the surface and wherein the working electrode and the counter electrode are formed using laser ablation to define the dimensions of the working electrode and the counter electrode.

13. The system of claim 9, further comprising a reference electrode on the surface of the substrate, the dielectric covering a portion of the reference electrode.

14. The system of claim 9, the anti-β-amyloid 42 antibody, anti-T-Tau antibody, and/or anti-P-Tau antibody being chemically functionalized to the surface of the working electrode coated with a 3-mercaptopropionic acid (MPA) monolayer.

15. The system of claim 10, wherein the sample comprises blood or serum.

16. A detection system for detecting β-amyloid 42, T-Tau, and/or P-Tau levels in a sample, the system comprising:
a sensor that includes a substrate, a working electrode formed on a surface of the substrate; a counter electrode formed on the surface of the substrate; a dielectric layer covering a portion of the working electrode and counter electrode and defining an aperture exposing other portions of the working electrode and counter electrode; and an anti-β-amyloid 42 antibody, anti- T-Tau antibody, and/or anti-P-Tau antibody functionalized or chemically functionalized to a surface of the exposed portion of the working electrode, the anti-β-amyloid 42 antibody, anti-T-Tau antibody, and/or anti-P-Tau antibody selectively binding to, respectively, β-amyloid 42, T-Tau, and/or P-Tau in a sample and the β-amyloid 42, T-Tau, and/or P-Tau once bound being detectable by measuring the current flow between the working electrode and counter electrode,
an equimolar potassium ferrocyanide/potassium ferricyanide redox solution that is applied to the working electrode for determining the quantity of β-amyloid 42, T-Tau, and/or P-Tau in the sample bound to, respectively, the anti-β-amyloid 42, anti-T-Tau, and/or anti-P-Tau antibody, and
a measuring device for applying voltage potentials to the working electrode and counter electrode and measuring the current flow between the working electrode and counter electrode.

17. The system of claim 16, wherein the working electrode and the counter electrode comprise metalized films, the metalized films are provided on the surface of the substrate by sputtering or coating the films on the surface and wherein the working electrode and the counter electrode are formed using laser ablation to define the dimensions of the working electrode and the counter electrode.

18. The system of claim 16, wherein the sample comprises blood or cerebrospinal fluid.
